(19) United States
(12) Patent Application Publication
(10) Pub. No.: US 2008/0247974 A1
(43) Pub. Date: Oct. 9, 2008

(54) Rooibos Extract with Increased Aspalathin Content, Process for the Preparation of Such a Rooibos Extract , and Cosmetic Agent Containing Such a Rooibos Extract

(75) Inventors: Sabine Gruner-Richter, Freising (DE); Frank Otto, Kranzberg (DE); Bernd Weinreich, Penzberg (DE)

Correspondence Address: THE WEBB LAW FIRM, P.C., 700 KOPPERS BUILDING, 436 SEVENTH AVENUE, PITTSBURGH, PA, 15219, US

(73) Assignee: RAPS GmbH & CO. KG, Kulmbach (DE)

(21) Appl. No.: 11/883,314

(22) PCT Filed: Jan. 27, 2006
(86) PCT No.: PCT/EP2006/000723

§ 371 Date: Apr. 22, 2008

(30) Foreign Application Priority Data
Jan. 31, 2005 (DE) 10 2005 004 438.7

(51) Int. Cl. A61K 8/97; A61Q 11/00; A61Q 5/00; A61Q 19/00; A61K 36/48

(52) U.S. Cl. 424/58; 424/74; 424/725

(57) ABSTRACT

The invention relates to a rooibos extract prepared from unfermented rooibos raw material by means of a solvent extraction, with an increased aspalathin content that is at least 5 wt %, cosmetic agents prepared from such a rooibos extract, and a process for the preparation of such a rooibos extract having an increased aspalathin content and preferably a reduced chlorophyll content.

The present invention relates to a novel rooibos extract, a process for the preparation of such a rooibos extract, as well as cosmetic agents containing such a rooibos extract. Preferably the rooibos extract according to the invention also has a greatly reduced chlorophyll content.

Rooibos, in German red bush, in Latin Aspalathus linearis, grows exclusively in South Africa and contains, as the only such type of plant in the world, the particularly highly antioxidatively acting substance aspalathin, which is a flavonoid. Rooibos is known to many people from red bush tea, since fermented and dried rooibos leaves are traditionally used as a tea or tea extract. The rooibos plant is free of alkaloids, contains in particular no caffeine, and compared for example to green tea has only a low tannin content, which means that red bush tea is also suitable for persons with a sensitive stomach.

Red bush tea extracts with an aspalathin content of not more than 1 to 3% are commercially available. For a better utilisation of the aforementioned antioxidative action of the aspalathin contained in rooibos, it would be desirable to be able to use a rooibos extract with a higher aspalathin content. At the same time it would be desirable if such an extract contained as low a content of chlorophyll as possible, since a rooibos extract with a high chlorophyll content leads to an undesirable discolouration when used in cosmetic agents.

The object of the present invention is accordingly to provide a rooibos extract with increased aspalathin content and preferably significantly reduced chlorophyll content, and a process for the preparation of such an improved rooibos extract, so that such a rooibos extract can also be used in cosmetic agents.

This object is achieved according to the invention by a rooibos extract prepared from unfermented rooibos raw material by means of solvent extraction, which has an aspalathin content of at least 5 wt % and a chlorophyll content of less than 0.4 wt %. An aspalathin content of at least 5 wt % is sufficiently high in order to ensure a medicinal effectiveness when the extract is used for example in cosmetic agents. The chlorophyll content of less than 0.4 wt %, preferably of less than 0.2 wt % and particularly preferably of less than 0.1 wt % prevents an undesirable discolouration of a product containing a rooibos extract according to the invention.

Preferably the aspalathin content of the rooibos extract according to the invention is more than 10 wt %, particularly more than 15 wt % or even more than 20 wt %. Rooibos extracts with such high aspalathin contents were not hitherto obtainable. The advantage of a high aspalathin content is seen in the fact that, with a given product, in order to achieve the same effectiveness the addition of a lower amount of rooibos extract is sufficient, which on account of the then likewise lower chlorophyll content also reduces the danger of an undesirable discolouration of the product. A further advantage of a high aspalathin content is based on the fact that, with a given amount of rooibos extract that is added to a product, the content of cosmetically active aspalathin is significantly increased. Aspalathin acts not only as a powerful antioxidant, but also as an anti-irritant and antimicrobial, which is advantageous especially when the extract according to the invention is used in cosmetic agents for hair, skin or mouth care or treatment.

Preferably the rooibos extract according to the invention contains even smaller amounts of chlorophyll than specified hereinbefore, and particularly preferably less than 0.15 wt %.

As already mentioned, the rooibos extract according to the invention is particularly suitable for use in cosmetic agents for the treatment or care of the skin, hair or also oral cavity. Agents, in particular cosmetic agents, that contain the rooibos extract according to the invention are therefore also covered by the present invention. The object mentioned in the introduction is accordingly also achieved by a cosmetic agent that contains 0.01 to 10 Wt % of the rooibos extract according to the invention. Such a cosmetic agent preferably exists in the form of a solution, an emulsion, or in the form of a gel. Examples of cosmetic agents according to the invention are a dental gel, an after-sun lotion, a coloured vanishing creme or also a lipstick.

If the cosmetic agent according to the invention is present in the form of an emulsion, this emulsion is preferably a cream. If a conventional cream base for cosmetic agents contains 0.1 wt % of the rooibos extract according to the invention, then the resultant cream has a demonstrable anti-irritant action in the so-called “half-side” test and after application to the skin for several days significantly reduces the transepidermal water loss (TEWL). Furthermore, such a cream provided with the extract according to the invention has a regeneration-supporting action on the skin. Apart from the powerful antioxidative action of the extract according to the invention, which has been confirmed by scientifically recognised tests such as ABTS, fentone, peroxy nitride, Rose Bengal, copper-induced Rose Bengal, xanthine/xanthine oxidase, the rooibos extract according to the invention also has an antimicrobial action, for example on the pathogens Escherichia Coli, Staphylococcus Aureus, Pseudomonas Aeroginosa, as well as in addition a fungicidal action, for example with respect to Aspergillus Niger.

In order to protect cosmetic agents containing the rooibos extract according to the invention against an undesirable discolouration also over a prolonged period, these cosmetic agents preferably contain 0.01 to 2 wt %, in particular 0.1 to 2 wt %, of an additive protecting against discolouration. Examples of such an additive are ascorbic acid, ascorbyl palmitate, α-tocopherol, α-tocopheryl acetate, tert.-butyl-hydroquinone, hydroquinone, propyl gallate, Na chlorate, Na EDTA, gallic acid, phytic acid, Rosemary acid, a carnosol acid-containing extract of Rosemary, or mixtures of these substances.

Alternatively it is possible to protect cosmetic agents that contain the rooibos extract according to the invention against discolouration by lowering the pH value to 4 or less.

The preparation of a rooibos extract according to the invention with an increased aspalathin content and preferably also a reduced chlorophyll content can be carried out particularly well by employing a special process, which is therefore also covered by the present invention. Accordingly, the object mentioned in the beginning is also achieved by a process for the preparation of a rooibos extract according to the invention, which comprises the following steps:
providing dried and comminuted, unfermented rooibos raw material,
extracting the supplied raw material with an extracting agent consisting of a mixture of ethanol and water for a predetermined extraction time at a temperature of 15° to 35° C.,
filtering the extract,
concentrating the filtered extract to dryness by evaporation under reduced pressure,
grinding the dried extract,
washing the ground extract with a solvent for a predetermined time,
filtering off the extract/solvent mixture and drying the resultant filter cake under reduced pressure until a predetermined residual content of solvent is obtained.

An important point for the process according to the invention is accordingly the provision of a suitable starting material consisting of dried and unfermented rooibos raw material. Normally the starting material is a mixture of leaves and stems of the Aspalathus linearis plant obtained during harvesting, though advantageously it is also possible to use only leaves as starting material. Preferably the moisture content of the supplied rooibos raw material is 4% or less, since a self-fermentation of the starting material is thereby prevented. If only leaves are used as starting material, the moisture content of the supplied rooibos raw material need only be 7% or less. A moisture content of 7% corresponds in this case (only leaves as starting material) to a moisture content of 4% in the case of a mixture of leaves and stems, since the stems dry more completely and therefore in the case of a mixture lead to a lower average moisture content of the dried mixture, i.e. the stems have in the mixture a significantly lower moisture content compared to the leaves.

According to a preferred embodiment of the process according to the invention, an ethanol/water mixture in a ratio of 80 to 20 is used as extracting agent. In this preferred embodiment of the process according to the invention the ratio of crude product to extracting agent is preferably 1 to 5, and the extraction step is preferably carried out at room temperature for a time of 1.5 to 3 hours. Other ethanol/water mixture ratios may however also be used, in which the ethanol fraction is higher, but may also be lower, and if necessary may be significantly lower.

The step of concentrating the filtered extract by evaporation is preferably carried out at a pressure of less than 100 mbar, and especially at a pressure of less than 30 mbar. The temperature in the concentration step is preferably at most 40° C.

The step of washing the ground extract with a solvent is preferably carried out with a ratio of extract to solvent of 1 to 5 for a period of 1.5 to 3 hours.

The predetermined residual content of solvent to be achieved by drying the filter cake of the extract/solvent mixture is preferably 20 ppm or less.

A particularly preferred embodiment of the process according to the invention for preparing the rooibos extract is described in more detail hereinafter. Unfermented and comminuted raw material (leaves and stems) of Aspalathus linearis carefully dried to a moisture content of less than 4% is used as starting material. This raw material is extracted with a mixture of ethanol and water in a ratio of 80 to 20 at 15° to 35° C. for 1.5 to 3 hours, the ratio of raw material to solvent being 1 to 5.

The extract obtained is concentrated by evaporation to dryness under reduced pressure, preferably at 30 mbar and at a temperature of at most 40° C. The dried extract is then ground and washed with ethyl acetate for a period of 1.5 to 3 hours at room temperature, the ratio of extract to solvent and wash agent being 1 to 5.

The mixture of ethyl acetate and the extract washed therewith is filtered after the washing stage, the filtrate is then discarded, and the filter cake is dried under reduced pressure until the measurable solvent residue in the filter cake is less than 20 ppm.

A fine, beige-green powder with an aspalathin content of at least 15 wt % is obtained, the obtained extract now containing 1 to at most 3% of the chlorophyll of the raw material.

A preparation Example is given hereinafter:

4 kg of a dried and comminuted rooibos raw material (leaves and stems) with a content of 3.7% of aspalathin are extracted with a mixture of 16 kg of ethanol and 4 kg of water for 90 minutes at 30° C. After filtering off the insoluble residue of Aspalathus linearis, the filtrate obtained is concentrated by evaporation to dryness at a reduced pressure of less than 100 mbar and at a temperature of 40° C. This crude extract is finely ground, 1.8 kg of ethyl acetate are added and the mixture is stirred for 90 minutes at room temperature. The mixture is then filtered and the filter cake is dried under reduced pressure of less than 100 mbar for 2 hours. 340 g of extract are obtained containing 16.2 wt % of aspalathin. The extract contains 0.058 wt % of chlorophyll, which corresponds to 0.9% of the chlorophyll content of the employed raw material.

A rooibos extract according to the invention prepared by the process described hereinbefore was used in various cosmetic agents according to the following Examples.

### EXAMPLE 1

### Dental Gel with the Following Composition

| Trade Name | CTFA | INCI | Amt. in % |
|---|---|---|---|
| Sorbitol | Sorbitol | Sorbitol | 40.000 |
| Sident S 9 | Silica | Silica | 12.000 |
| Glycerol | Glycerol | Glycerol | 10.000 |
| Sident S 22 | Silica | Silica | 10.000 |
| Tagat L 2 | PEG-20 Glyceryl Laurate | PEG-20 Glyceryl Laurate | 5.000 |
| Extrapon Sage GW | Glycerol/Water/Sage | Glycerol/Water/Sage | 1.000 |
| | Extract/Sodium | Extract/Sodium | |
| | Benzoate/Potassium | Benzoate/Potassium | |
| | Sorbate | Sorbate | |
| Extrapon Camomile GW | Glycerol/Water/Matri-caria | Glycerol/Water/Matri-caria | 1.000 |
| | Extract/Lactic Acid/Sodium | Extract/Lactic Acid/Sodium | |
| | Benzoate/Potassium | Benzoate/Potassium | |
| | Sorbate | Sorbate | |
| Rooibos | Rooibus (Leaf) Extract | Aspalathus Linearis | 0.200 |
| Sodium Saccharin | Sodium Saccharinate | Sodium Saccharinate | 0.100 |
| Blanose 7 M F | Cellulose Gum | Cellulose Gum | 0.100 |

| | |
|---|---|
| Water, Preservative, Aroma Substance | to 100 |

### EXAMPLE 2

### After-Sun Lotion with the Following Composition

| Trade Name | CTFA | INCI | Amt. in % |
|---|---|---|---|
| Miglyol 812 | Caprylic/Capric Triglyceride | Caprylic/Capric Triglyceride | 6.000 |
| Alcohol, denat. | SD-Alcholol 39 C | Alcohol denat. | 5.000 |
| Propylene glycol | Propylene Glycol | Propylene Glycol | 5.000 |
| Cetiol SN | Cetearyl Isononanoate | Cetearyl Isononanoate | 4.000 |
| Cetiol V | Decyl Oleate | Decyl Oleate | 2.500 |
| Tego Care 450 | Polyglyceryl-3- | Polyglyceryl-3- | 2.250 |
| | Methylglucose Distearate | Methylglucose Distearate | |
| Abil 100 | Dimethicone | Dimethicone | 0.500 |
| Pemulen TR 1 | Acrylates/C10-30 Alkyl | Acrylates/C10-30 Alkyl | 0.200 |
| | Acrylate Crosspolymer | Acrylate Crosspolymer | |
| Rooibos | Rooibus (Leaf) Extract | Aspalathus Linearis | 0.150 |
| Sodium Hydroxide Pellets | Sodium Hydroxide | Sodium Hydroxide | 0.020 |

| | |
|---|---|
| Water, Preservative, Paraffin Oil | to 100 |

### EXAMPLE 3

### Coloured Vanishing Creme with the Following Composition

| Trade Name | CTFA | INCI | Amt. in % |
|---|---|---|---|
| Arlatone 983 | PEG-5 Glyceryl Stearate | PEG-5 Glyceryl Stearate | 4.000 |
| Stearic Acid | Stearic Acid | Stearic Acid | 0.500 |
| Tegin SE | Glyceryl Stearate SE | Glyceryl Stearate SE | 1.200 |
| Lannette 16 | Cetyl Alcohol | Cetyl Alcohol | 2.000 |
| Abil 350 | Dimethicone | Dimethicone | 1.000 |
| Tegosoft OS | Octyl Stearate | Octyl Stearate | 3.000 |
| Phenova | Phenoxyethanol/ | Phenoxyethanol/ | 0.500 |
| | Methyl p-hydroxybenzoic acid/ | Methyl p-hydroxybenzoic acid/ | |
| | Ethyl p-hydroxybenzoic acid/ | Ethyl p-hydroxybenzoic acid/ | |
| | Propyl p-hydroxybenzoic acid/ | Propyl p-hydroxybenzoic acid/ | |
| | Butyl p-hydroxybenzoic acid/ | Butyl p-hydroxybenzoic acid/ | |
| | Isobutyl p-hydroxybenzoic acid | Isobutyl p-hydroxybenzoic acid | |
| AV 50 TSI | Titanium Dioxide/Isocetyl | Titanium Dioxide/Isocetyl | 3.000 |
| | Stearoyl Stearate/Avocado | Stearoyl Stearate/Avocado | |
| | Oil Unsaponifiables/ | Oil Unsaponifiables/ | |
| | Triethoxy | Triethoxy | |
| | Caprylyslilane/Water | Caprylyslilane/Water | |
| BYO-12 (yellow) | Iron Oxide/Isopropyl | Iron Oxide/Isopropyl | 0.300 |
| | Titanium Triisostearate | Titanium Triisostearate | |
| BRO-12 (red) | Iron Oxide/Isopropyl | Iron Oxide/Isopropyl | 0.300 |
| | Titanium Triisostearate | Titanium Triisostearate | |
| BBO-12 (black) | Iron Oxide/Isopropyl | Iron Oxide/Isopropyl | 0.100 |
| | Titanium Triisostearate | Titanium Triisostearate | |
| GMS/MM3 | Mica/Magnesium Myristate | Mica/Magnesium Myristate | 0.100 |
| Propylene Glycol | Propylene Glycol | Propylene Glycol | 5.000 |
| Panthenol | Panthenol | Panthenol | 1.000 |
| Rooibos | Rooibus (Leaf) Extract | Aspalathus Linearis | 0.100 |
| Ascorbic Acid | Ascorbic Acid | Ascorbic Acid | 0.200 |
| Sodium Bisulfite | Sodium Hydrogen Sulfite | Sodium Hydrogen Sulfite | 0.200 |
| Solution 39% | | | |
| Citric Acid | Citric Acid | Citric Acid | 0.100 |
| Water | Water | Aqua | to 100 |

### EXAMPLE 4

### Lipstick with the Following Composition

| Trade Name | CTFA | INCI | Amt. in % |
|---|---|---|---|
| Castor Oil | Castor Oil | Ricinus Communis | 46.700 |
| Beeswax | Beeswax | Cera Alba | 10.000 |
| Eutanol G | Octyldodecanol | Octyldodecanol | 10.000 |
| Candelilla Wax | Candelilla Wax | Candelilla Cera | 6.500 |
| Tegosoft SH | Stearyl Heptanoate | Stearyl Heptanoate | 6.500 |
| Ozocerite | Ozocerite | Ozocerite | 5.000 |
| Jojoba Oil | Jojoba Oil | Buxus Chinensis | 5.000 |
| Isopropyl | Isopropyl Lanoate | Isopropyl Lanoate | 5.000 |
| Lanoate | | | |
| Carnauba Wax | Carnauba Wax | Carnauba | 3.500 |
| Vitamin E | Tocopheryl Acetate | Tocopheryl Acetate | 0.500 |
| Acetate | | | |
| Bisabolol | Bisabolol | Bisabolol | 0.200 |
| Rooibos | Rooibus (Leaf) | Aspalathus Linearis | 0.100 |
| | Extract | | |
| Aroma | Aroma | Aroma | 1.000 |
| | | | 100.000 |

1-20. (canceled)

21: A rooibos extract, wherein the rooibos extract is prepared from unfermented rooibos raw material by a solvent extraction and has an aspalathin content of at least 5 wt %, as well as a chlorophyll content of less than 0.4 wt %.

22: The rooibos extract according to claim 21, wherein the aspalathin content is more than 10 wt %.

23: The rooibos extract according to claim 21, wherein the aspalathin content is more than 15 wt %.

24: A rooibos extract according to claim 21, wherein the chlorophyll content is less than 0.2 wt %.

25: The cosmetic agent, wherein the cosmetic agent comprises 0.01 to 10 wt % of a rooibos extract according to claim 21.

26: The cosmetic agent according to claim 25, wherein the cosmetic agent is in the form of a solution, an emulsion or in the form of a gel.

27: The cosmetic agent according to claim 25, wherein the cosmetic agent is used for the treatment and/or care of the skin, hair or oral flora.

28: The cosmetic agent according to claim 27, comprising:
a) a rooibos extract prepared from unfermented rooibos raw material by a solvent extraction, wherein the extract has an aspalathin content of at least 5 wt %, as well as a chlorophyll content of less than 0.4 wt %; and
b) 0.01 to 2 wt % of an additive protecting against coloration.

29: The cosmetic agent according to claim 28, wherein the additive is ascorbic acid, ascorbyl palmitate, a-tocopherol, a-tocopheryl acetate, tert.-butyl-hydroquinone, hydroquinone, propyl gallate, Na chlorate, Na EDTA, gallic acid, phytic acid, Rosemary acid, a carnosol acid-containing extract of Rosemary, or a mixture of these substances.

30: A process for the preparation of a rooibos extract according to claim 21, comprising the following steps:
providing dried and comminuted, unfermented rooibos raw material,
extracting the supplied raw material with an extracting agent consisting of a mixture of ethanol and water for a predetermined extraction time,
filtering the extract,
concentrating the filtered extract to dryness by evaporation under reduced pressure,
grinding the dried extract,
washing the ground extract with a solvent for a predetermined time, and
filtering off the extract/solvent mixture and drying the resultant filter cake under reduced pressure until a predetermined residual content of solvent is obtained.

31: The process according to claim 30, wherein the moisture content of the supplied rooibos raw material is 4% or less.

32: The process according to claim 30, wherein the extracting agent consists of ethanol and water in a ratio of 80:20.

33: The process according to claim 32, wherein the ratio of raw material to extracting agent is 1:5.

34: The process according to claim 33, wherein the extraction step is carried out at a temperature of 15° to 35° C.

35: The process according to claim 34, wherein the predetermined extraction time is 1.5 to 3 hours.

36: The process according to claim 30, wherein the step of concentrating the filtered extract by evaporation is carried out at a pressure of less than 100 mbar, preferably at a pressure of 30 mbar or less.

37: The process according to claim 36, wherein the step of concentrating the filtered extract by evaporation is carried out at a temperature of at most 40° C.

38: The process according to claim 30, wherein the step of washing the ground extract is carried out with a solvent in a ratio of extract to solvent of 1:5.

39: The process according to claim 38, wherein the step of washing the ground extract is carried out with a solvent for a time of 1.5 to 3 hours.

40: The process according to claim 30, wherein the predetermined residual content of solvent is 20 ppm or less.
